(19) United States
(12) Patent Application Publication
(10) Pub. No.: US 2011/0086761 A1
(43) Pub. Date: Apr. 14, 2011

(54) COMPOSITIONS AND METHODS TO CONTROL OOMYCETE FUNGAL PATHOGENS

(75) Inventors: Norman R. Pearson, Carmel, IN (US); Lei Liu, Carmel, IN (US); Robert J. Ehr, Indianapolis, IN (US); John M. Atkinson, Zionsville, IN (US)

(73) Assignee: DOW AGROSCIENCES LLC, Indianapolis, IN (US)

(21) Appl. No.: 12/903,708

(22) Filed: Oct. 13, 2010

Related U.S. Application Data
(60) Provisional application No. 61/251,037, filed on Oct. 13, 2009.

(51) Int. Cl. A01N 59/20; A61K 31/519; A01N 43/60; A01N 43/40; A01N 43/54; A01N 43/00; A01N 37/10; A01N 47/10; A01N 47/40; A01N 33/18; A01N 37/18; A01N 43/36; A01N 43/38; A01N 43/84; A61K 31/44; A01N 37/44; A01N 43/50; A61K 31/5375; A01N 43/78; A01N 43/32; A01N 35/02; A01P 13/00; A01P 3/00; A01P 7/04

(52) U.S. Cl. 504/121; 514/259.1; 504/136; 504/130; 504/139; 504/143; 504/144; 504/141; 514/741; 514/599; 424/637; 514/425; 514/414; 514/256; 514/229.2; 514/349; 514/538; 514/390; 514/616; 514/237.5; 514/625; 514/365; 514/385; 514/357; 514/434; 514/478; 424/633; 514/693

(57) ABSTRACT

The present invention relates to fungicidal compositions and their use for controlling oomycete pathogen induced disease or diseases in one or more plants.

### CROSS REFERENCE TO RELATED APPLICATIONS

This application claims the benefit of U.S. Provisional Patent Application Ser. No. 61/251,037, filed Oct. 13, 2009, which is expressly incorporated by reference herein.

### FIELD OF THE INVENTION

This invention relates to methods and compositions suitable for controlling oomycete fungal plant pathogens.

BACKGROUND AND SUMMARY OF THE INVENTION

During the asexual life cycle of a number of oomycete pseudo-fungi, such as Phytophthora infestans, the cause of late blight of potatoes, and Plasmopara viticola, which causes downy mildew of grapes, spores are produced by the pathogen called sporangia. Under suitable conditions, the contents of sporangia form additional spores called zoospores. Zoospores have flagella and are capable of swimming in water, i.e. they are motile. Zoospores serve as major infection agents by swimming to and encycsting near the stomata of a plant or other suitable place on the leaf, stem, root, seed or tuber for infecting the plant. On foliage, the stomata are then entered into by germ tubes from the germinating cysts or in some cases the germ tube from the encysted zoospore can directly pentrate the plant or root surface.

Past research has identified some chemicals known to attract zoospores. These zoospore attractants may generally be described as a substance or compound that causes a chemotactic response by a zoospore. Examples of zoospore attractants chemicals are disclosed in the article “Fatty acids, aldehydes and alcohols as attractants for zoospores of Phytophthora palmivora” in Nature, volume 217, page 448, by Cameron and Carlile. Further examples of zoospore attractants may be found in the articles “Biology of Phytophthora zoospores” in Phytopathology, volume 60, pages 1128-1135 by Hickman and “Chemotactic response of zoospores of five species of Phytophthora” in Phytopathology, volume 63, pages 1511-1517 by Khew and Zentmeyer. The disclosures of each of the above mentioned articles are expressly incorporated by reference herein. Generally, these zoospore attractant chemicals or substances are produced by the root region of plants and may enhance the infection process in the rhizosphere by enabling the zoospores to locate a point for infection. It is possible that plant foliage or specific sites on the foliage also produce substances that are attractive to zoospores.

Substances can be tested for their ability to attract zoospores through chemotaxis using a variety of published methods, including those employing capillary tubes that emanate the substance to be tested. Such methods are broadly applicable and are described in various publications, such as:
Donaldson, S. P. and J. W. Deacon. 1993. New Phytologist, 123: 289-295.
Tyler, B. M., M-H. Wu, J-M. Wang, W. Cheung and P. F. Morris. 1996. Applied and Environmental Microbiology, 62: 2811-2817.
Khew, K. I. and G. A. Zentmeyer. 1973. Phytopathology, 63: 1511-1517.

Generally, compounds to be tested for their ability to attract zoospores through chemotaxis must have sufficient water solubility or, if of low water solubility, they must be in a suitable physical form to allow sufficient wetting and release of the test compound. Suitable physical forms could include properly emulsified samples dissolved in water-insoluble solvents or solids that have been wet or dry milled with appropriate surfactants such that the samples have adequate wetting and dispersion in water and are of a suitable size (<10 microns) to test in a capillary system.

The present disclosure provides new methods and compositions of controlling oomycete fungal plant pathogens. The inventive composition typically comprises a composition suitable for controlling oomycete fungi capable of producing zoospores, the composition including an agriculturally effective amount of one or more fungicides, at least one of a zoospore attractant and a zoospore attractant derivative, one or more binders and, optionally, other inert formulation ingredients, that offers improved disease control.

### DETAILED DESCRIPTION OF THE INVENTION

The present invention relates to fungicidal compositions and their use for controlling oomycete pathogen induced disease or diseases in one or more plants. The inventive methods comprise contacting a plant at risk of being diseased from an oomycete pathogen that produces zoospores with a composition comprising an effective amount of a fungicide, at least one of a zoospore attractant and a zoospore attractant derivative, one or more binders and optionally, other inert formulation ingredients. Alternatively, the composition of the present invention may be comprised of differing zoospore attractants and zoospore attractant derivatives as well as a mixture of differing fungicides.

While not wishing to be bound by any theory it is believed that placing a fungicide particle in close proximity to a zoospore attractant or a zoospore attractant derivative in order to create a point source of a mobile, water soluble, zoospore attractant and the fungicide, may beneficially enhance the effectiveness of the composition. In the composition of the present invention, the binder serves to provide a matrix, a coating or acts like an adhesive whereby the fungicide and zoospore attractant or zoospore attractant derivative are held in close proximity to one another. The composition of the present invention may provide improved disease control in spray applications when compared to separately tank-mixing each individual component in the spray mixture. Additionally, a broader range of fungicides may be used, including fungicides that have limited redistribution on the plant surface.

While not wishing to be bound by any theory it is believed that using a zoospore attractant or zoospore attractant derivative and one or more binders and optionally, other inert formulation ingredients, may enhance the effectiveness of zoospore active fungicides such as thiocarbamates such as mancozeb, maneb, zineb, thiram, propineb, or metiram; copper-based fungicides such as copper hydroxide, copper oxychloride, or Bordeaux mixture; phthalimide fungicides such as captan or folpet; amisulbrom; strobilurins such as azoxystrobin, trifloxystrobin, picoxystrobin, kresoxim-methyl, pyraclostrobin, fluoxastrobin, and others; famoxadone; fenamidone; metalaxyl; mefenoxam; benalaxyl; cymoxanil; propamocarb; dimethomorph; flumorph; mandipropamid; iprovalicarb; benthiavalicarb-isopropyl; valiphenal, valiphenate; zoxamide; ethaboxam; cyazofamid; fluopicolide; fluazinam; chlorothalonil; dithianon; fosetyl-AL, phosphorous acid; tolylfluanid, aminosulfones such as 4-fluorophenyl (1S)-1-({[(1R,S)-(4-cyanophenyl)ethyl]sulfonyl}methyl)-propylcarbamate or triazolopyrimidine compounds such as ametoctradin and those shown by Formula I:

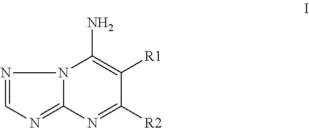

wherein R1 is ethyl, 1-octyl, 1-nonyl, or 3,5,5-trimethyl-l-hexyl and R2 is methyl, ethyl, 1-propyl, 1-octyl, trifluoromethyl, or methoxymethyl.

Useful zoospore attractants may vary depending upon the type of plant, the fungal pathogen and environmental conditions. Typical zoospore attractants may include C4-C8 aldehydes, C4-C8 carboxylic acids, C3-C8 amino acids, C4-C8 alcohols, flavones, flavanes and iso-flavones, amines, sugars, C4-C8 ketones, stilbenes, benzoins, benzoates, benzophenones, acetophenones, biphenyls, coumarins, chromanones, tetralones and anthraquinones.

Suitable zoospore attractant C4-C8 carboxylic acids may include isocaproic acid, isovaleric acid, valeric acid, caproic acid, cinnamic acid, and their C1-C8 ester derivatives which can release the attractant molecules under suitable conditions. Suitable zoospore attractant C3-C8 amino acids may include asparagine, L-aspartate (aspartic acid), L-glutamate, L-glutamine, L-asparagine, L-alanine, arginine, leucine, and methionine. Suitable zoospore attractant C4-C8 alcohols may include isoamyl alcohol.

Suitable zoospore attractant flavones and iso-flavones may include cochliophilin A (5-hydroxy-6,7-methylenedioxyflavone), 4′-hydroxy-5,7-dihydroxyflavone, daidzein (7,4′-dihydroxyisoflavone), genistein (5,7,4′-trihydroxyisoflavone), 5,4′-dihydroxy-3,3′-dimethoxy-6,7-methylenedioxyflavone, prunetin (5,4′-dihydroxy-7-methoxyisoflavone), N-trans-feruloyl-4-O-methyldopamine, daidzin and genistin which are carbohydrate conjugates of daidzein and genistein, respectively, biochanin A, formononetin, and isoformononetin.

Suitable zoospore attractant amines may include isoamyl amine and amide derivatives thereof.

Suitable zoospore attractant sugars may include naturally occurring mono- and di-saccharides such as D-glucose, D-mannose, L-fucose, maltose, D-fructose, and sucrose.

Suitable zoospore attractant C4-C8 ketones may include 4-methyl-2-pentanone, 3-methyl-2-pentanone, 3,3-dimethyl-2-butanone and their derivatives such as hydrazones, acylhydrazones, oximes, nitrones, imines, enamines, bisulfite addition compounds, ketals, and condensation products with urea which can release the attractant molecules under suitable conditions.

Suitable zoospore attractant C4-C8 aldehydes may include isovaleraldehyde, 2-methylbutyraldehyde, valeraldehyde, isobutyraldehyde, butyraldehyde, 4-methylpentanal, 3,3-dimethylbutyraldehyde, 3-methylthiobutyraldehyde, 2-cyclopropylacetaldehyde, 3-methylcrotonaldehyde, 2-ethylcrotonaldehyde, crotonaldehyde, 2-methylcrotonaldehyde, furfural (2-furaldehyde), 2-thiophenecarboxaldehyde, 2-ethylbutyraldehyde, cyclopropanecarboxaldehyde, 2,3-dimethylvaleraldehyde, 2-methylvaleraldehyde, tetrahydrofuran-3-carboxaldehyde, and cyclopentanecarboxaldehyde and their derivitives such as hydrazones, acylhydrazones, oximes, nitrones, animals, imines, enamines, bisulfite addition compounds, acetals, and condensation products with urea which can release the attractant molecules under suitable conditions.

Preferred zoospore attractants are isovaleraldehyde, 2-methylbutyraldehyde, valeraldehyde, isobutyraldehyde, butyraldehyde, 4-methylpentanal and 3,3-dimethylbutyraldehyde.

In addition to zoospore attractants, zoospore attractant derivatives may also be used in compositions of the present invention for purposes such as controlled release of the zoospore attractant molecule. Zoospore attractant derivatives are chemical compounds generally made or derived from zoospore attractant molecules. Zoospore attractant derivatives may be used in combination with zoospore attractants or independently. Suitable zoospore attractant derivatives such as hydrazone derivatives of zoospore attractants may be used for controlled release of a zoospore attractant when the derivative comes into contact with water on a plant surface or the area adjacent to the plant. Controlled release of the zoospore attractant from a zoospore attractant derivative may allow for more efficient use of the zoospore attractant by providing longer residuality of the zoospore attractant on the plant surface whereas use of a zoospore attractant alone might lead to rapid loss of it due to evaporation or water wash-off and thereby the attractant effect may be reduced. Examples of hydrazone derivative technology are included in PCT Patent Application No. WO2006016248 and the article entitled “Controlled release of volatile aldehydes and ketones by reversible hydrazone formation—‘classical’ profragrances are getting dynamic” by Levrand et al. published in Chemical Communications (Cambridge, United Kingdom) (2006) on pages 2965-2967 (ISSN: 1359-7345). The disclosure of each of the above references is hereby expressly incorporated by reference herein.

Preferred zoospore attractant derivatives of the present invention are isophthalic acid dihydrazide bis-isovaleraldehyde hydrazone, terephthalic acid dihydrazide bis-isovaleraldehyde hydrazone, the semicarbazone of isovaleraldehyde, carbohydrazide bis-isovaleraldehyde hydrazone, oxalyl dihydrazide bis-isovaleraldehyde hydrazone, malonic acid dihydrazide bis-isovaleraldehyde hydrazone, succinic acid dihydrazide bis-isovaleraldehyde hydrazone, glutaric acid dihydrazide bis-isovaleraldehyde hydrazone, adipic acid dihydrazide bis-isovaleraldehyde hydrazone, pimelic acid dihydrazide bis-isovaleraldehyde hydrazone, sebacic acid dihydrazide bis-isovaleraldehyde hydrazone, acetic acid hydrazide isovaleraldehyde hydrazone, propionic acid hydrazide isovaleraldehyde hydrazone, butyric acid hydrazide isovaleraldehyde hydrazone, valeric acid hydrazide isovaleraldehyde hydrazone, caproic acid hydrazide isovaleraldehyde hydrazone, heptanoic acid hydrazide isovaleraldehyde hydrazone, octanoic acid hydrazide isovaleraldehyde hydrazone, nonanoic acid hydrazide isovaleraldehyde hydrazone, decanoic acid hydrazide isovaleraldehyde hydrazone, dodecanoic acid hydrazide isovaleraldehyde hydrazone, tetradecanoic acid hydrazide isovaleraldehyde hydrazone, hexadecanoic acid hydrazide isovaleraldehyde hydrazone, stearic acid hydrazide isovaleraldehyde hydrazone, 4-phenylsemicarbazone of isovaleraldehyde, benzoic acid hydrazide isovaleraldehyde hydrazone and compounds derived from the condensation of isovaleraldehdye and urea.

Binders are components of the present invention that may associate the fungicide or fungicides and the zoospore attractant or zoospore attractant derivative in close proximity to one another. In one embodiment, the binder or binders may serve to provide a coating or a matrix that allows particles of the different components to become closely associated with or bound to one another such that an aggregate particle containing particles of fungicide and particles of zoospore attractant or particles of zoospore attractant derivative may be formed which may then serve as a point source for release of the various components on or near the plant.

Suitable binders of the present invention include, but are not limited to, proteins, polypeptides, peptides, amino acids, polysaccharides, lignins, gelatins, gums, celluloses, chitosans, natural latexes, wood rosin and modified derivatives and combinations thereof, and man-made polymers such as polyolefins such as polyallene, polybutadiene, polyisoprene, and poly(substituted butadienes) such as poly(2-t-butyl-1,3-butadiene), poly(2-chlorobutadiene), poly(2-chloromethyl butadiene), polyphenylacetylene, polyethylene, chlorinated polyethylene, polypropylene, polybutene, polyisobutene, polycyclopentylethylene and polycyclolhexylethylene, polystyrene, poly(alkylstyrene), poly (substituted styrene), poly(biphenyl ethylene), poly(1,3-cyclohexadiene), polycyclopentadiene, polyacrylates including polyalkylacrylates and polyarylacrylates, polyacrylonitrile, polymethacrylates including polyalkylmethacrylates and polyarylmethacrylates, polylactates, polyvinyl pyrrolidones, polydisubstituted esters such as poly(di-n-butylitaconate), and poly(amylfumarate), polyvinylethers such as poly(butoxyethylene) and poly(benzyloxyethylene), poly(methyl isopropenyl ketone), polyvinyl chloride, polyvinylidene chloride, polyvinyl alcohol, polyvinyl acetate, polyvinyl carboxylate esters such as polyvinyl propionate, polyvinyl butyrate, polyvinyl caprylate, polyvinyl laurate, polyvinyl stearate, polyvinyl benzoate, polyurethanes, epoxy resins and the like, and modified derivatives, combinations and co-polymers thereof, and inorganic compounds such as metal salts and metal oxides and their combinations with other binders. The man-made polymers may be used directly or as particle dispersions in water commonly known as latexes.

Preferred binders are proteins, such as egg albumin, man-made latexes, partially hydrolyzed polyvinyl alcohols, co-polymers of partially hydrolyzed polyvinyl alcohols, polyvinyl pyrrolidones, co-polymers of polyvinyl pyrrolidones, modified starches, chitosan, metal salts and metal oxides and mixtures thereof.

Latexes are generally defined as stable dispersions of polymer microparticles in aqueous medium. Latexes may be natural or synthetic. Latex as found in nature is a milky, sap-like fluid within many plants that coagulates on exposure to air. It is a complex emulsion in which proteins, alkaloids, starches, sugars, oils, tannins, resins, and gums are found. Man-made latex rubber is made by polymerizing a monomer or monomers that has been emulsified with surfactants in a water system or by dispersing a powdered polymer in water.

Latexes that are preferred binders in compositions of the present invention are acrylic, vinylacrylic, methacrylic, vinylmethacrylic and styrene-butadiene latexes and mixtures, co-polymers and derivatives thereof. The acrylic and methacrylic containing latexes comprise ester groups derived from C1-C20 alcohols.

Inerts are defined as carriers, wetting agents, adjuvants, dispersing agents, stabilizers, rheology additives, freezing-point depressants, antimicrobial agents, crystallization inhibitors, water and other suitable components known in the art.

Compositions of the present invention may be prepared by suitably dispersing in water, in the appropriate particle sizes, the components of the present invention and then drying the resulting dispersion, for example by spray drying, to provide a dry, wettable powder. The drying may be achieved by spray drying, drum drying or by other methods known to those skilled in the art. The dry or wettable powder may be further processed into other formulation types such as dispersible granules (DG), suspension concentrates (SC) or oil dispersions (OD) using known methods.

Compositions of the present invention may contain one or more fungicides that comprise 10-90% by weight of the formulation, one or more binders comprising 1-20% by weight of the formulation, one or more of a zoospore attractant and zoospore attractant derivative comprising 1-25% by weight of the formulation and one or more inert ingredients comprising 1-90% by weight of the formulation.

The aforementioned compositions of the present invention have been found to be particularly effective in controlling diseases caused by the pathogens Phytophthora infestans, Plasmopara viticola, Phytophthora capsici, and Pseudoperonospora cubensis. Other pathogens that may also be controlled for a variety of plants such as, but not limited to, tomatoes, potatoes, peppers, grapes, cucurbits, lettuce, beans, sorghum, corn, citrus, turf grasses, pecans, apples, pears, hops, and crucifiers include, but are not limited to, Bremia lactucae, Phytophthora phaseoli, Phytophthora nicotiane var. parasitica, Sclerospora graminicola, Sclerophthora rayssiae, Phytophthora palmivora, Phytophthora citrophora, Sclerophthora macrospora, Sclerophthora graminicola, Phytophthora cactorum, Phytophthora syringe, Pseudoperonospora humuli, and Albugo candida.

The effective amount of the composition of the present invention to be employed in controlling or preventing disease development on plants often depends upon, for example, the type of plants, the stage of growth of the plants, severity of environmental conditions, the fungal pathogen and application conditions. Typically, a plant in need of fungal protection, control or elimination is contacted with the composition of the present invention diluted in a carrier such as water that will provide an amount of zoospore attractant or zoospore attractant derivative from about 0.1 to about 5000 ppm, preferably from about 1 to about 1000 ppm of an attractant or zoospore attractant derivative and one or more fungicides in an amount from about 1-40,000 ppm, preferably from about 10-20,000 ppm of one or more fungicides. The contacting may be in any effective manner.

For example, any part of the plant, e.g., leaves or stems may be contacted with the composition of the present invention containing the zoospore attractant or zoospore attractant derivative in mixture with effective rates of a fungicide or fungicides. Such compositions could be applied to foliage, blossoms, fruit, and/or stems of plants and that in various instances they could also be effective for improving disease control when applied to seeds, roots, tubers or in the general rhizosphere in which the plant is growing.

The aforementioned compositions of the present invention may be applied to the plant foliage or the soil or area adjacent to the plant. Additionally, the compositions of the present invention may be mixed with or applied with any combination of agricultural active ingredients such as herbicides, insecticides, bacteriocides, nematocides, miticides, biocides, termiticides, rodenticides, molluscides, arthropodicides, fertilizers, modifiers of plant physiology or structure, and pheromones.

### Preparations

Representative Preparation of a Composition of the Present Disclosure via Spray-Drying (Sample 57 in Table 3 Below)

A solution of 69 g of water, 0.83 g of 15% aqueous polyvinyl alcohol (Celvol 205) and 0.75 g of sodium lignosulfonate (Borresperse Na) was stirred well with a dispersing stirrer and then treated with 7.8 g of 24% isophthalic acid dihydrazide bis-isovaleraldehyde hydrazone (compound A in Table 1 below) dispersed in water (contains 2.2% of wetting and dispersing surfactants and was previously ball-milled to a particle size of ca. 2.5-3.0 microns (d (0.5)). The mixture was well stirred for 10 minutes and then was treated with 0.57 g of 55% aqueous UCAR 379G latex and finally 20.8 g of 85% technical DITHANE® WP (mancozeb, registered trademark of Dow AgroSciences, LLC). The resulting mixture was stirred a further 10 minutes and then was homogenized on a Silverson Homogenizer at 5000-5500 rpm for 15 minutes (a few drops of Breakthru Antifoam 9903 were added). The resulting 100 g mixture containing 25 g of non-volatile components was spray dried on a Model B-190 Buchi laboratory spray dryer: liquid feed rate 300 mL/hr (syringe pump used), inlet temperature 134-136° C., outlet temperature 88-93° C., 600 mL/min nozzle air flow, 5 bar nozzle pressure and an aspirator vacuum pump was used at the end of the process (part of Buchi spray dryer). The spray dried solid was collected in a cyclone collector to furnish 10 g of gold colored solid with an average particle size of 10.9 microns (d(0.5) as measured in water on a MasterSizer 2000 particle size analyzer). This method or slight modifications thereof were used to prepare the samples listed in Table 3.

Table 1 below lists the zoospore attractant derivatives used to prepare the compositions of the present disclosure which are shown in Table 3.

TABLE 1
Identification of Zoospore Attractants Derivatives

| Compound | Structure |
|---|---|
| A | 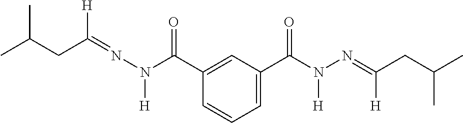 |
| B | 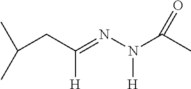 |
| C (condensation product of urea and isovalderaldehyde) | 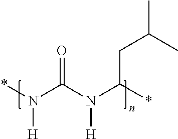 |
| D | 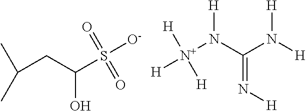 |
| E | 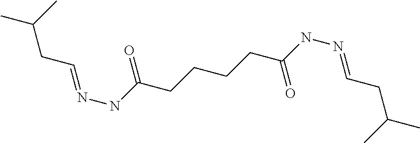 |
| F | 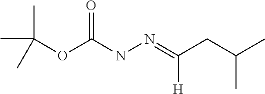 |

Compounds A, B, E and F were prepared by heating a mixture of the corresponding hydrazide or bis-hydrazide starting material and a molar excess of isovaleraldehyde at reflux in ethanol solvent until the the hydrazide or bis-hydrazide was completely converted into the mono- or bis-hydrazone of isovaleraldehyde. The products were then isolated and purified by methods commonly used by those skilled in the art, to provide the desired compounds that were characterized by proton NMR spectroscopy and by CHN elemental analysis. Compounds A, E and F were separately ball-milled in water with surfactants to provide aqueous suspension concentrates of the respective zoospore attractant derivative with an average particle size distribution (d (0.5)) of less than 10 microns. Compound B was sufficiently soluble in water to be used without milling

Sample C was prepared by mechanically stirring a mixture of isovaleraldehyde, water and a catalytic amount of 85% phosphoric acid, heating it to approximately 40° C. and then treating it quickly with a solution of 2 molar equivalents of urea dissolved in water. The resulting solution exothermed to approximately 60° C. as a heavy, white solid formed. The very viscous mixture was stirred for one hour at ambient temperature and the solid present was collected by filtration, washed with water and vacuum oven dried to constant weight. This material was ball-milled in water with surfactants to provide an aqueous suspension concentrate of the zoospore attractant derivative.

Compound D was prepared by dissolving the sodium bisulfite addition compound of isovaleraldehdye in water and treating it at room temperature with an equimolar amount of a solution of aminoguanidine hydrochloride salt in water. The white solid that crystallized over the next several days was collected, washed with ethanol and vacuum dried to provide a white solid that was sufficiently soluble in water to be used without milling Table 2 below provides a description of the binders used to prepare the samples of the present disclosure that are listed in Table 3. The mancozeb used was 85% DITHANE® (trademark of Dow AgroSciences, LLC) technical manufactured by Dow AgroSciences, LLC. The dimethomorph was technical grade and was ball-milled in water with surfactants prior to use.

TABLE 2
Binder Descriptions for Compositions in Table 3.

| Binder¹ | Description¹ |
|---|---|
| latex A | UCAR ® 379G latex² |
| latex B | Neocar ® 820 latex |
| latex C | Neocar ® 2300 latex |
| latex D | UCAR ® 627 latex² |
| latex E | UCAR ® DT250 latex² |
| latex F | CP620 NA latex³ |
| latex G | LXC 8476 NA latex³ |
| latex H | XU 30792 latex³ |
| latex I | UCAR ® 418 latex |
| EA | albumin from chicken egg white, Grade II; Sigma |
| PVA | Celvol ® 205 polyvinyl alcohol |
| NaLS | sodium lignosulfonate dispersant - |
| | Borresperse ® NA |
| CaLS | calcium lignosulfonate dispersant - |
| | Borresperse ® CA |
| Fe2O3 | iron oxide, 500M micron milled powder; |
| | Magnetics Intl. Inc |
| nano Fe2O3 | iron oxide, nano-sized; Sigma-Aldrich |
| CuO | copper (II) oxide, unmilled; Sigma-Aldrich |
| nano CuO | copper (II) oxide, nano-sized; Sigma-Aldrich |
| ZnO | zinc oxide; Nanox ® 500; Elementis |
| Agrimer ® VA6 | vinylpyrrolidone/vinylacetate co-polymer, |
| | 4/6 ratio; ISP |
| Agrimer ® VA3E | vinylpyrrolidone/vinylacetate co-polymer, |
| | 3/7 ratio; ISP |
| Agrimer ® VA7E | vinylpyrrolidone/vinylacetate co-polymer, |
| | 7/3 ratio; ISP |
| AB starch | starch, unmodified; Lykeby Culinar |
| chitosan | de-acetylated chitin, low molecular weight; |
| | Sigma-Aldrich |

¹Agrimer is registered trademark of International Specialty Products; UCAR is a registered trademark of The Dow Chemical Company; Neocar is a registered trademark of Arkema Inc.; Celvol is a registered trademark of Sekisui Specialty Chemicals America LLC; Borresperse is a registered trademark of Borregaard LignoTech; Nanox is a registered trademark of Elementis;
²Available from Arkema Inc.
³Available from The Dow Chemical Company.

TABLE 3
Identification of Compositions of the Present Disclosure

| Sample | | Fungicide | Wt %, Zoosp. Attrac. | | | | | | 3% NaLS |
|---|---|---|---|---|---|---|---|---|---|
| Number | Fungicide | wt % | Derivative | | Wt %, Binder 1 | | Wt %, Binder 2 | | Added |
| 1 | mancozeb | 60.0% | 15% | cmpd. A | 10% | EA | 3% | Fe2O3 | |
| 2 | mancozeb | 60.0% | 15% | cmpd. A | 10% | EA | 3% | PVA | |
| 3 | mancozeb | 60.0% | 15% | cmpd. A | 10% | latex B | | | |
| 4 | mancozeb | 60.0% | 15% | cmpd. A | 10% | latex A | | | |
| 5 | mancozeb | 60.0% | 15% | cmpd. C | 10% | EA | 3% | Fe2O3 | |
| 6 | mancozeb | 60.0% | 15% | cmpd. C | 10% | EA | 3% | PVA | |
| 7 | mancozeb | 60.0% | 15% | cmpd. B | 10% | EA | 3% | Fe2O3 | |
| 8 | mancozeb | 60.0% | 15% | cmpd. B | 10% | EA | 3% | PVA | |
| 9 | mancozeb | 60.0% | 15% | cmpd. D | 10% | EA | 3% | Fe2O3 | |
| 10 | mancozeb | 68.0% | 17% | cmpd. A | 3% | PVA | | | |
| 11 | mancozeb | 68.0% | 17% | cmpd. C | 3% | PVA | | | |
| 12 | mancozeb | 60.0% | 15% | cmpd. D | 10% | EA | 3% | PVA | |
| 13 | mancozeb | 69.0% | 7.5% | cmpd. A | 5% | latex A | 3% | PVA | yes |
| 14 | mancozeb | 71.0% | 7.5% | cmpd. A | 2.5% | latex A | 3% | PVA | yes |
| 15 | mancozeb | 69.2% | 7.3% | cmpd. C | 4.8% | latex A | 3% | PVA | yes |
| 16 | mancozeb | 70.7% | 7.4% | cmpd. C | 2.5% | latex A | 3% | PVA | yes |
| 17 | mancozeb | 58.8% | 15% | cmpd. A | 9.8% | EA | 5% | PVA | |
| 18 | mancozeb | 59.1% | 15% | cmpd. A | 9.9% | EA | 1.5% | PVA | yes |
| 19 | mancozeb | 66.4% | 3.50% | cmpd. A | 11.0% | EA | 3.4% | PVA | yes |
| 20 | mancozeb | 63.5% | 7.5% | cmpd. A | 10.6% | EA | 3.2% | PVA | yes |
| 21 | mancozeb | 63.2% | 7.5% | cmpd. A | 10.5% | EA | 3.2% | Fe2O3 | yes |
| 22 | mancozeb | 63.6% | 7.5% | cmpd. A | 10.6% | EA | 3.2% | ZnO | yes |
| 23 | mancozeb | 66.4% | 3.75% | cmpd. A | 11.1% | EA | 3.2% | ZnO | yes |
| 24 | mancozeb | 66.4% | 3.75% | cmpd. A | 11.1% | EA | 3.3% | Fe₂O₃ | yes |
| 25 | mancozeb | 63.2% | 7.5% | cmpd. A | 10.5% | EA | 3.2% | nano Fe₂O₃ | yes |
| 26 | mancozeb | 66.4% | 3.75% | cmpd. A | 11.1% | EA | 3.3% | nano Fe₂O₃ | yes |
| 27 | mancozeb | 57.6% | 15% | cmpd. A | 9.6% | EA | 2.9% | nano Fe₂O₃ | yes |
| 28 | mancozeb | 57.6% | 15% | cmpd. A | 9.6% | EA | 2.9% | ZnO | yes |
| 29 | mancozeb | 57.6% | 15% | cmpd. A | 9.6% | EA | 2.9% | nano CuO | yes |
| 30 | mancozeb | 63.2% | 7.5% | cmpd. A | 10.5% | EA | 3.2% | nano CuO | yes |
| 31 | mancozeb | 66.4% | 3.75% | cmpd. A | 11.1% | EA | 3.3% | nano CuO | yes |
| 32 | mancozeb | 69.2% | 7.3% | cmpd. A | 4.8% | latex B | 2.9% | PVA | yes |
| 33 | mancozeb | 70.7% | 7.3% | cmpd. A | 2.5% | latex B | 3.0% | PVA | yes |
| 34 | mancozeb | 70.0% | 7.5% | cmpd. A | 5.0% | EA | 1.5% | PVA | yes |
| 35 | mancozeb | 75.7% | 3.75% | cmpd. A | 2.5% | EA | 0.75% | PVA | yes |
| 36 | mancozeb | 65.5% | 7.5% | cmpd. A | 10.0% | EA | 1.5% | PVA | yes |
| 37 | mancozeb | 69.7% | 3.75% | cmpd. A | 10.0% | EA | .75% | PVA | yes |
| 38 | mancozeb | 69.0% | 3.75% | cmpd. A | 10.0% | EA | 1.5% | PVA | yes |
| 39 | mancozeb | 69.5% | 7.5% | cmpd. A | 5% | latex A | 0.5% | PVA | yes |
| 40 | mancozeb | 69.5% | 7.5% | cmpd. C | 5% | latex A | 0.5% | PVA | yes |
| 41 | mancozeb | 69.9% | 7.5% | cmpd. B | 5% | EA | 1.5% | PVA | yes |
| 42 | mancozeb | 73.3% | 3.75% | cmpd. B | 5% | EA | 1.5% | PVA | yes |
| 43 | mancozeb | 68.7% | 7.5% | cmpd. B | 5% | latex A | 3% | PVA | yes |
| 44 | mancozeb | 70.9% | 7.5% | cmpd. B | 5% | latex A | 0.5% | PVA | yes |
| 45 | mancozeb | 65.8% | 7.5% | cmpd. B | 10% | EA | 1.5% | PVA | yes |
| 46 | mancozeb | 66.3% | 7.5% | cmpd. B | 10% | EA | 0.75% | PVA | yes |
| 47 | mancozeb | 70.0% | 7.5% | cmpd. A | 5% | latex A | 1.5% | PVA | yes |
| 48 | mancozeb | 72.1% | 7.5% | cmpd. A | 2.5% | latex A | 1.5% | PVA | yes |
| 49 | mancozeb | 57.6% | 15% | cmpd. A | 9.6% | EA | 2.9% | CuO | yes |
| 50 | mancozeb | 57.6% | 15% | cmpd. A | 9.6% | EA | 2.9% | CaCO₃ | yes |
| 51 | mancozeb | 64.0% | 7.5% | cmpd. C | 10.53% | EA | 3% | nano Fe2O3 | yes |
| 52 | mancozeb | 58.8% | 15% | cmpd. A | 9.60% | EA | 1.4% | Fe2O3 | yes |
| 53 | mancozeb | 64.5% | 7.5% | cmpd. A | 10.53% | EA | 1.6% | Fe2O3 | yes |
| 54 | mancozeb | 59.8% | 15% | cmpd. A | 9.9% | latex A | 0.5% | PVA | yes |
| 55 | mancozeb | 71.3% | 7.5% | cmpd. A | 5.0% | latex A | | | yes |
| 56 | mancozeb | 73.5% | 7.5% | cmpd. A | 1.26% | latex A | | | yes |
| 57 | mancozeb | 70.7% | 7.5% | cmpd. A | 1.25% | latex A | 0.5% | PVA | yes |
| 58 | mancozeb | 71.7% | 7.5% | cmpd. A | 1.25% | latex A | 3% | PVA | yes |
| 59 | mancozeb | 72.9% | 7.5% | cmpd. A | 3% | PVA | | | yes |
| 60 | mancozeb | 73.1% | 7.5% | cmpd. A | 1.25% | latex A | 1.5% | PVA | yes |
| 61 | mancozeb | 70.3% | 7.5% | cmpd. A | 5% | latex B | 1% | PVA | yes |
| 62 | mancozeb | 70.3% | 7.5% | cmpd. A | 5% | latex C | 1% | PVA | yes |
| 63 | mancozeb | 70.3% | 7.5% | cmpd. A | 5% | latex A | 1% | PVA | yes |
| 64 | mancozeb | 70.3% | 7.5% | cmpd. A | 5% | latex D | 1% | PVA | yes |
| 65 | mancozeb | 71.1% | 7.5% | cmpd. B | 5% | latex A | 1% | PVA | yes |
| 66 | mancozeb | 71.1% | 7.5% | cmpd. B | 5% | latex B | 1% | PVA | yes |
| 67 | mancozeb | 71.1% | 7.5% | cmpd. B | 5% | latex C | 1% | PVA | yes |
| 68 | mancozeb | 71.1% | 7.5% | cmpd. B | 5% | latex D | 1% | PVA | yes |
| 69 | mancozeb | 71.1% | 7.5% | cmpd. B | 5% | latex E | 1% | PVA | yes |
| 70 | mancozeb | 70.2% | 7.5% | cmpd. A | 5% | latex E | 1% | PVA | yes |
| 71 | mancozeb | 70.8% | 9.00% | cmpd. A | 0.8% | PVA | | | yes |
| 72 | mancozeb | 71.2% | 9.00% | cmpd. A | 0.82% | latex A | 0.7% | PVA | yes |
| 73 | mancozeb | 71.4% | 7.5% | cmpd. B | 2.5% | latex A | 3% | PVA | yes |
| 74 | mancozeb | 72.5% | 7.5% | cmpd. B | 1.25% | latex A | 3% | PVA | yes |
| 75 | mancozeb | 73.5% | 7.5% | cmpd. B | 3% | PVA | | | yes |
| 76 | mancozeb | 70.6% | 7.5% | cmpd. B | 5.0% | latex A | 1.5% | PVA | yes |
| 77 | mancozeb | 72.7% | 7.5% | cmpd. B | 2.5% | latex A | 1.5% | PVA | yes |
| 78 | mancozeb | 73.7% | 7.5% | cmpd. B | 1.25% | latex A | 1.5% | PVA | yes |
| 79 | mancozeb | 74.6% | 7.5% | cmpd. B | 1.25% | latex A | 0.5% | PVA | yes |
| 80 | mancozeb | 71.8% | 7.5% | cmpd. B | 5.0% | latex A | | | yes |
| 81 | mancozeb | 75.0% | 7.5% | cmpd. B | 1.25% | latex A | | | yes |
| 82 | mancozeb | 72.4% | 7.5% | cmpd. A | 0.76% | latex A | 3% | PVA | yes |
| 83 | mancozeb | 73.5% | 7.5% | cmpd. A | 0.76% | latex A | 1.5% | PVA | yes |
| 84 | mancozeb | 74.8% | 7.5% | cmpd. A | 1.5% | latex A | | | yes |
| 85 | mancozeb | 74.8% | 7.5% | cmpd. A | 1.5% | PVA | | | yes |
| 86 | mancozeb | 72.9% | 7.5% | cmpd. A | 1.25% | latex B | 1.5% | PVA | yes |
| 87 | mancozeb | 72.9% | 7.5% | cmpd. A | 1.25% | latex E | 1.5% | PVA | yes |
| 88 | mancozeb | 72.9% | 7.5% | cmpd. A | 1.25% | latex D | 1.5% | PVA | yes |
| 89 | mancozeb | 71.9% | 7.5% | cmpd. A | 2.50% | latex E | 1.5% | PVA | yes |
| 90 | mancozeb | 73.7% | 7.5% | cmpd. B | 1.25% | latex B | 1.5% | PVA | yes |
| 91 | mancozeb | 73.7% | 7.5% | cmpd. B | 1.25% | latex D | 1.5% | PVA | yes |
| 92 | mancozeb | 73.7% | 7.5% | cmpd. B | 1.25% | latex E | 1.5% | PVA | yes |
| 93 | mancozeb | 69.6% | 7.5% | cmpd. A | 1.25% | latex A | 1.5% | PVA | yes |
| 94 | mancozeb | 66.6% | 7.5% | cmpd. A | 5.0% | latex E | 1.5% | PVA | yes |
| 95 | mancozeb | 69.6% | 7.5% | cmpd. A | 1.25% | latex E | 1.5% | PVA | yes |
| 96 | mancozeb | 66.6% | 7.5% | cmpd. A | 5.0% | latex B | 1.5% | PVA | yes |
| 97 | mancozeb | 69.6% | 7.5% | cmpd. A | 1.25% | latex B | 1.5% | PVA | yes |
| 98 | mancozeb | 66.5% | 7.5% | cmpd. A | 5.0% | latex A | 1.5% | PVA | yes |
| 99 | mancozeb | 69.5% | 7.5% | cmpd. A | 1.25% | latex D | 1.5% | PVA | yes |
| 100 | mancozeb | 66.5% | 7.5% | cmpd. A | 5.0% | latex D | 1.5% | PVA | yes |
| 101 | mancozeb | 70.1% | 7.5% | cmpd. B | 1.25% | latex A | 1.5% | PVA | yes |
| 102 | mancozeb | 70.1% | 7.5% | cmpd. B | 1.25% | latex D | 1.5% | PVA | yes |
| 103 | mancozeb | 69.7% | 7.5% | cmpd. B | 1.25% | latex F | 1.5% | PVA | yes |
| 104 | mancozeb | 70.3% | 7.5% | cmpd. B | 1.25% | latex F | 1.5% | PVA | yes |
| 105 | mancozeb | 69.7% | 7.5% | cmpd. A | 1.25% | latex G | 1.5% | PVA | yes |
| 106 | mancozeb | 70.3% | 7.5% | cmpd. B | 1.25% | latex G | 1.5% | PVA | yes |
| 107 | mancozeb | 69.7% | 7.5% | cmpd. A | 1.25% | latex H | 1.5% | PVA | yes |
| 108 | mancozeb | 70.3% | 7.5% | cmpd. B | 1.25% | latex H | 1.5% | PVA | yes |
| 109 | mancozeb | 69.7% | 7.5% | cmpd. A | 1.25% | latex I | 1.5% | PVA | yes |
| 110 | mancozeb | 70.3% | 7.5% | cmpd. B | 1.25% | latex I | 1.5% | PVA | yes |
| 111 | mancozeb | 67.2% | 7.5% | cmpd. E | 1.25% | latex A | 1.5% | PVA | yes |
| 112 | mancozeb | 66.2% | 7.5% | cmpd. E | 2.5% | latex A | 1.5% | PVA | yes |
| 113 | mancozeb | 70.3% | 7.5% | cmpd. E | 1.5% | PVA | | | yes |
| 114 | mancozeb | 69.3% | 7.5% | cmpd. E | 1.25% | latex B | 1.5% | PVA | yes |
| 115 | mancozeb | 68.9% | 7.5% | cmpd. E | 1.25% | latex D | 1.5% | PVA | yes |
| 116 | mancozeb | 68.9% | 7.5% | cmpd. E | 1.25% | latex C | 1.5% | PVA | yes |
| 117 | mancozeb | 69.2% | 7.5% | cmpd. E | 1.25% | latex E | 1.5% | PVA | yes |
| 118 | mancozeb | 68.8% | 7.5% | cmpd. C | 1.25% | latex A | 1.5% | PVA | yes |
| 119 | mancozeb | 68.8% | 7.5% | cmpd. C | 1.25% | latex B | 1.5% | PVA | yes |
| 120 | mancozeb | 68.8% | 7.5% | cmpd. C | 1.25% | latex D | 1.5% | PVA | yes |
| 121 | mancozeb | 68.8% | 7.5% | cmpd. C | 1.25% | latex C | 1.5% | PVA | yes |
| 122 | mancozeb | 68.8% | 7.5% | cmpd. C | 1.25% | latex E | 1.5% | PVA | yes |
| 123 | mancozeb | 73.2% | 7.5% | cmpd. F | 1.25% | latex D | 1.5% | PVA | yes |
| 124 | mancozeb | 73.1% | 7.5% | cmpd. F | 1.25% | latex A | 1.5% | PVA | yes |
| 125 | mancozeb | 73.1% | 7.5% | cmpd. F | 1.25% | latex H | 1.5% | PVA | yes |
| 126 | dimethomorph | 80.5% | 7.5% | cmpd. A | 1.25% | latex A | 1.0% | PVA | no (1.5%) |
| 127 | dimethomorph | 81.1% | 7.5% | cmpd. B | 1.25% | latex A | 1.0% | PVA | no (1.5%) |
| 128 | mancozeb | 74.8% | 7.5% | cmpd. A | 1.5% | Agrimer VA6 | | | yes |
| 129 | mancozeb | 73.5% | 7.5% | cmpd. A | 3.0% | Agrimer VA6 | | | yes |
| 130 | mancozeb | 73.5% | 7.5% | cmpd. A | 3.0% | AB starch | | | yes |
| 131 | mancozeb | 73.5% | 7.5% | cmpd. A | 3.0% | chitosan | | | yes |
| 132 | mancozeb | 73.5% | 7.5% | cmpd. A | 3.0% | sodium alginate | | | yes |
| 133 | mancozeb | 73.5% | 7.5% | cmpd. A | 3.0% | asparagine | | | yes |
| 134 | mancozeb | 73.5% | 7.5% | cmpd. A | 3.0% | Agrimer VA3 E | | | yes |
| 135 | mancozeb | 73.5% | 7.5% | cmpd. A | 3.0% | Agrimer VA7 E | | | yes |
| 136 | mancozeb | 60.0% | 15% | cmpd. A | 10% | EA | 3% | PVA | |
| 137 | mancozeb | 60.0% | 15% | cmpd. A | 3% | PVA | | | no - 10% CaLS |
| 138 | mancozeb | 60.0% | 15% | cmpd. B | 10% | EA | 3% | PVA | no - 1.4% CaLS |
| 139 | mancozeb | 60.0% | 15% | cmpd. A | 10% | EA | 3% | PVA | |

The following examples were carried out in greenhouse and growth chamber experiments.

Grapes (Vitis vinifera cv Carignane), tomatoes (Lycopersicon esculentum cv Outdoor Girl), and cucumbers (Cucumis sativus cv Bush Pickle Hybrid #901261) were grown from seed in 5 cm by 5 cm pots containing MetroMix™ growth medium (Scotts, Marysville, Ohio). Plants were raised in greenhouses with supplementary light sources on a 14 hour photoperiod and maintained at 20-26° C. Healthy plant growth was maintained through regular application of dilute liquid fertilizer solution containing a complete range of nutrients. When plants were in the 2-4 true leaf stage of growth, plants with uniform growth were selected for spray application and trimmed Grapes were trimmed to have two true leaves; cucumbers were trimmed to have one true leaf.

Candidate samples and DITHANE® DG NT (mancozeb; trademark of Dow AgroSciences LLC) were formulated in water such that mancozeb rates delivered from each formulation were 25, 12.5, 6.25 and 3.12 ppm. The dilute spray solutions were applied using an automated high volume rotary sprayer fitted with two 6128-1/4 JAUPM spray nozzles (Spraying Systems, Wheaton, Ill.) operated at 20 psi and configured to provide thorough coverage of both leaf surfaces. Each treatment was replicated 3 or 4 times. Sprayed plants were randomized after spray application.

Plants were inoculated 18-24 hours after formulations were applied. Inoculum of Phytophthora infestans (PHYTIN) was prepared from cultures grown in the dark on solid rye seed agar. When abundant sporangia were present, deionized water was added to the plates and then brushed lightly to dislodge sporangia. Inoculum of Plasmopara viticola (PLASVI) was produced by placing infected grape plants in a dew chamber overnight to promote sporulation. Leaves with abundant sporangia were placed in deionized water and brushed lightly to dislodge sporangia. Similarly, inoculum of Pseudoperonospora cubensis (PSPECU) was produced by placing infected cucumber plants in a dew chamber overnight to promote sporulation. Leaves with abundant sporangia were placed in deionized water and brushed lightly to dislodge sporangia.

Sporangium concentration of each pathogen was adjusted to 80,000 sporangia per ml. Plants were inoculated by applying a fine mist with a low pressure (5 psi) compressed air sprayer at a volume of approximately 200 ml per 80 pots of grapes, tomatoes, or cucumbers. Plants were incubated for 24 hours in a dew chambers maintained at about 16-22° C., depending on the plant and disease. Tomatoes and cucumbers were then transferred to well-lighted growth chambers maintained at 20° C. for subsequent disease development. Grapes were transferred to a greenhouse on a 14 hour photoperiod and maintained at 24-26° C. for symptom development. Visual assessments of the level of disease on tomatoes and cucumbers were made 4-7 days after inoculation when the level of disease in untreated but inoculated check plants reached 75-95% disease. When symptoms were clearly visible on grape leaves, they were moved into a dew chamber to allow sporulation. Visual assessments of the level of disease were then made based on the percent of the lower leaf surface covered by sporulating lesions. Results of these tests are shown in Tables 4 through 6.

TABLE 4
Percent disease on plants sprayed with Dithane DGNT or
co-formulations of mancozeb plus zoospore attractant
derivatives and infected with Phytophthora infestans,
Plasmopara viticola or Pseudoperonospora cubensis.

| | Sample number | | | | | |
|---|---|---|---|---|---|---|
| | Late blight | | Grape downy | | | |
| | of tomato - | | mildew - | | Cucumber downy | |
| | PHYTIN | | PLASVI | | mildew - PSPECU | |
| | Mancozeb conc | | | | | |
| | 25 ppm | 12.5 ppm | 25 ppm | 12.5 ppm | 6.25 ppm | 3.12 ppm |
| | Dithane DG NT | | | | | |
| | 61 | 80 | 0 | 47 | 28 | 60 |
| 20 | 14 | 54 | 5 | 15 | 2 | 42 |
| 18 | 16 | 56 | 2 | 3 | 19 | 17 |
| 2 | 16 | 79 | 17 | 22 | 15 | 7 |
| 13 | 41 | 61 | 1 | 17 | 4 | 11 |
| 4 | 24 | 55 | 5 | 63 | 13 | 12 |
| 136 | 21 | 76 | 2 | 12 | 2 | 15 |
| 137 | 30 | 56 | 0 | 15 | 15 | 7 |
| 138 | 12 | 29 | 0 | 9 | 5 | 28 |
| 139 | 29 | 68 | 4 | 7 | 15 | 30 |
| 8 | 16 | 65 | 3 | 22 | 0 | 23 |
| 10 | 50 | 58 | 7 | 18 | 8 | 25 |

| | | | |
|---|---|---|---|
| Untreated check | 95 | 95 | 77 |

TABLE 5
Percent disease on plants sprayed with Dithane DG
NT or co-formulations of mancozeb plus zoospore attractant
derivatives and infected with either Phytophthora
infestans or Plasmonara viticola.

| | Late blight of tomato - | | Grape downy mildew | |
|---|---|---|---|---|
| | PHYTIN | | PLASVI | |
| | Mancozeb conc | | | |
| Sample number | 25 ppm | 12.5 ppm | 25 ppm | 12.5 ppm |
| Dithane DGNT | 35 | 89 | 6 | 15 |
| 13 | 44 | 70 | 12 | 7 |
| 14 | 53 | 54 | 0.5 | 3 |
| 21 | 36 | 60 | 0 | 1.5 |
| 22 | 16 | 72 | 0 | 8 |
| 23 | 22 | 64 | 0 | 4 |
| 24 | 16 | 54 | 1 | 1 |
| 25 | 41 | 70 | 4 | 4 |
| 26 | 29 | 70 | 0 | 1 |
| 27 | 76 | 63 | 0.5 | 2 |
| 28 | 46 | 69 | 0 | 1 |
| 1 | 53 | 79 | 1 | 9 |
| 4 | 41 | 69 | 37 | 66 |

| | | |
|---|---|---|
| Untreated check | 95 | 95 |

TABLE 6
Percent disease on cucumber plants sprayed with Dithane DGNT plus
GF-2004 or co-formulations of mancozeb plus zoospore attractant derivatives
and infected with Pseudoperonospora cubensis.

| | Grape downy mildew - | | Cucumber downy | |
|---|---|---|---|---|
| | PLASVI | | mildew - PSPECU | |
| | Mancozeb conc | | | |
| Sample number | 25 ppm | 12.5 ppm | 12.5 ppm | 6.25 ppm |
| Dithane DG NT plus | 22 | 57 | 9 | 23 |
| 10% SC of | | | | |
| Compound A¹ | | | | |
| 15 | 9 | 15 | 1 | 2 |
| 8 | 1 | 18 | 1 | 37 |
| 48 | 0 | 12 | 1 | 5 |
| 47 | 4 | 50 | 17 | 37 |
| 44 | 2 | 23 | 4 | 10 |
| 40 | 3 | 3 | 5 | 18 |
| 13 | 4 | 17 | 1 | 7 |
| 45 | 1 | 42 | 12 | 10 |
| 14 | 4 | 12 | 1 | 20 |
| 42 | 0 | 10 | 9 | 7 |
| 41 | 2 | 2 | 1 | 1 |
| 16 | 4 | 15 | 3 | 4 |
| 43 | 5 | 14 | 1 | 9 |
| 46 | 6 | 8 | 0 | 9 |
| 138 | 0 | 9 | 0 | 8 |
| 39 | 2 | 7 | 1 | 9 |
| 37 | 0 | 3 | 1 | 17 |
| 33 | 0 | 7 | 0 | 3 |
| 32 | 1 | 5 | 2 | 15 |

| | | |
|---|---|---|
| Untreated check | 95 | 95 |

¹Dithane DG NT was tank-mixed with compound A (formulated as a 10% SC) at a mancozeb to compound A weight ratio of 9.3 to 1, respectively.

While the invention has been described with respect to a limited number of embodiments, the specific features of one embodiment should not be attributed to other embodiments of the invention. No single embodiment is representative of all aspects of the invention. In some embodiments, the compositions or methods may include numerous compounds or steps not mentioned herein. In other embodiments, the compositions or methods do not include, or are substantially free of, any compounds or steps not enumerated herein. Variations and modifications from the described embodiments exist. Finally, any number disclosed herein should be construed to mean approximate, regardless of whether the word “about” or “approximately” is used in describing the number. The appended embodiments and claims intend to cover all those modifications and variations as falling within the scope of the invention.

1. A composition suitable for controlling oomycete fungi capable of producing zoospores, the composition including:
an agriculturally effective amount of a fungicide;
at least one of a zoospore attractant and a zoospore attractant derivative; and
a binder.

2. The composition of claim 1, further including an inert ingredient.

3. The composition of claim 1, wherein the fungicide is selected from the group consisting of mancozeb, maneb, zineb, thiram, propineb, metiram, copper hydroxide, copper oxychloride, Bordeaux mixture, captan, folpet, amisulbrom, azoxystrobin, trifloxystrobin, picoxystrobin, kresoxim-methyl, famoxadone, fenamidone, metalaxyl, mefenoxam, benalaxyl, cymoxanil, propamocarb, dimethomorph, flumorph, mandipropamid, iprovalicarb, benthiavalicarb-isopropyl, valiphenal, valiphenalate, zoxamide, ethaboxam, cyazofamid, fluopicolide, fluazinam, chlorothalonil, dithianon, tolylfluanid, 4-fluorophenyl (1S)-1-({[(1R,S)-(4-cyanophenyl)ethyl]sulfonyl}methyl)propyl-carbamate, ametoctradin and compounds of Formula 1: 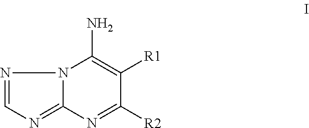
wherein R1 is ethyl, 1-octyl, 1-nonyl, or 3,5,5-trimethyl-l-hexyl; and R2 is methyl, ethyl, 1-propyl, 1-octyl, trifluoromethyl, and methoxymethyl.

4. The composition of claim 3 wherein the fungicide is selected from mancozeb, chlorothanil, cymoxanil, metalaxyl, mefenoxam, dimethomorph, mandipropamid, propamocarb, fluopicolide, fluazinam, metiram, propineb, fenamidone and cyazofamid.

5. The composition of claim 4, wherein the fungicide is mancozeb.

6. The composition of claim 1, wherein the zoospore attractant is a C4-C8 aldehyde selected from the group consisting of isovaleraldehyde, 2-methylbutyraldehyde, valeraldehyde, isobutyraldehyde, butyraldehyde, 4-methylpentanal, 3,3-dimethylbutyraldehyde, 3-methylthiobutyraldehyde, 2-cyclopropylacetaldehyde, 3-methylcrotonaldehyde, 2-ethylcrotonaldehyde, crotonaldehyde, 2-methylcrotonaldehyde, furfural (2-furaldehyde), 2-thiophenecarboxaldehyde, 2-ethylbutyraldehyde, cyclopropanecarboxaldehyde, 2,3-dimethylvaleraldehyde, 2-methylvaleraldehyde, tetrahydrofuran-3-carboxaldehyde, and cyclopentanecarboxaldehyde.

7. The composition of claim 6, wherein the zoospore attractant is isovaleraldehyde, 2-methylbutyraldehdye, isobutyraldehyde, 3,3-dimethyl-butyraldehyde, cyclopropylacetaldehyde, 3-methyl-2-butenaldehyde and valeraldehyde.

8. The composition of claim 7, wherein the zoospore attractant is isovaleraldehyde.

9. The composition of claim 1, wherein the zoospore attractant derivative is one of isophthalic acid dihydrazide bis-isovaleraldehyde hydrazone, terephthalic acid dihydrazide bis-isovaleraldehyde hydrazone, the semicarbazone of isovaleraldehyde, carbohydrazide bis-isovaleraldehyde hydrazone, oxalyl dihydrazide bis-isovaleraldehyde hydrazone, malonic acid dihydrazide bis-isovaleraldehyde hydrazone, succinic acid dihydrazide bis-isovaleraldehyde hydrazone, glutaric acid dihydrazide bis-isovaleraldehyde hydrazone, adipic acid dihydrazide bis-isovaleraldehyde hydrazone, pimelic acid dihydrazide bis-isovaleraldehyde hydrazone, sebacic acid dihydrazide bis-isovaleraldehyde hydrazone, acetic acid hydrazide isovaleraldehyde hydrazone, propionic acid hydrazide isovaleraldehyde hydrazone, butyric acid hydrazide isovaleraldehyde hydrazone, valeric acid hydrazide isovaleraldehyde hydrazone, caproic acid hydrazide isovaleraldehyde hydrazone, heptanoic acid hydrazide isovaleraldehyde hydrazone, octanoic acid hydrazide isovaleraldehyde hydrazone, nonanoic acid hydrazide isovaleraldehyde hydrazone, decanoic acid hydrazide isovaleraldehyde hydrazone, dodecanoic acid hydrazide isovaleraldehyde hydrazone, tetradecanoic acid hydrazide isovaleraldehyde hydrazone, hexadecanoic acid hydrazide isovaleraldehyde hydrazone, stearic acid hydrazide isovaleraldehyde hydrazone, 4-phenylsemicarbazone of isovaleraldehyde and benzoic acid hydrazide isovaleraldehyde hydrazone.

10. The composition of claim 1, wherein the binder is selected from the group consisting of proteins, albumins, natural latexes, man-made latexes, partially hydrolyzed polyvinyl alcohols, co-polymers of partially hydrolyzed polyvinyl alcohols, polyvinyl pyrrolidones, co-polymers of polyvinyl pyrrolidones, metal oxides, metal salts, gelatins, chitosans, starches, carbohydrates, amino acids and mixtures and derivatives thereof.

11. The composition of claim 1, wherein the binder is one of natural and man-made latex.

12. The composition of claim 11, wherein the man-made latex is one of an acrylic, a vinylacrylic, a methacrylic, a vinylmethacrylic, a styrene-butadiene latex and mixtures, co-polymers and derivatives thereof.

13. The composition of claim 1, wherein the binder is a 87-89% hydrolyzed polyvinyl alcohol having an average molecular weight range of 31,000 to 50,000.

14. The composition of claim 2, wherein a dry powder is formed by one of dispersing and dissolving the fungicide, the at least one of the zoospore attractant and the zoospore attractant derivative, the binder and the inert ingredient in water and then drying a resulting dispersion.

15. A method for controlling plant diseases caused by oomycete fungal pathogens including the steps of:
providing a formulation including the composition of claim 1;
diluting the formulation containing the composition of claim 1 in a suitable agricultural carrier; and
applying the formulation to at least one of the plant, an area adjacent to the plant, plant foliage, blossoms, stems, fruits, soil, seeds, germinating seeds, roots, liquid and solid growth media, and hydroponic growth solutions.

16. A method of controlling at least one of insects, plants diseases and weeds including the steps of:
providing a formulation including the composition of claim 1, and
applying an agriculturally effective amount of the formulation in mixture with at least one formulation of agricultural active ingredients and nutrients to at least one of a plant, plant foliage, blossoms, stems, fruits, an area adjacent to the plant, soil, seeds, germinating seeds, roots, liquid and solid growth media, and hydroponic growth solutions.

17. An aggregate particle including:
an agriculturally effective amount of a fungicide adapted to control plant diseases caused by oomycete fungal pathogens;
at least one of a zoospore attractant and a zoospore attractant derivative; and
a binder adapted to closely associate the at least one of the zoospore attractant and zoospore attractant derivative and the fungicide to form the aggregate particle.

18. The aggregate particle of claim 17, further including an inert ingredient.

19. The aggregate particle of claim 17 wherein the fungicide is selected from mancozeb, chlorothanil, cymoxanil, metalaxyl, mefenoxam, dimethomorph, mandipropamid, propamocarb, fluopicolide, fluazinam, metiram, propineb, fenamidone and cyazofamid.

20. The aggregate particle of claim 17, wherein the zoospore attractant is isovaleraldehyde.

21. The aggregate particle of claim17, wherein the zoospore attractant derivative is one of isophthalic acid dihydrazide bis-isovaleraldehyde hydrazone, terephthalic acid dihydrazide bis-isovaleraldehyde hydrazone, the semicarbazone of isovaleraldehyde, carbohydrazide bis-isovaleraldehyde hydrazone, oxalyl dihydrazide bis-isovaleraldehyde hydrazone, malonic acid dihydrazide bis-isovaleraldehyde hydrazone, succinic acid dihydrazide bis-isovaleraldehyde hydrazone, glutaric acid dihydrazide bis-isovaleraldehyde hydrazone, adipic acid dihydrazide bis-isovaleraldehyde hydrazone, pimelic acid dihydrazide bis-isovaleraldehyde hydrazone, sebacic acid dihydrazide bis-isovaleraldehyde hydrazone, acetic acid hydrazide isovaleraldehyde hydrazone, propionic acid hydrazide isovaleraldehyde hydrazone, butyric acid hydrazide isovaleraldehyde hydrazone, valeric acid hydrazide isovaleraldehyde hydrazone, caproic acid hydrazide isovaleraldehyde hydrazone, heptanoic acid hydrazide isovaleraldehyde hydrazone, octanoic acid hydrazide isovaleraldehyde hydrazone, nonanoic acid hydrazide isovaleraldehyde hydrazone, decanoic acid hydrazide isovaleraldehyde hydrazone, dodecanoic acid hydrazide isovaleraldehyde hydrazone, tetradecanoic acid hydrazide isovaleraldehyde hydrazone, hexadecanoic acid hydrazide isovaleraldehyde hydrazone, stearic acid hydrazide isovaleraldehyde hydrazone, 4-phenylsemicarbazone of isovaleraldehyde and benzoic acid hydrazide isovaleraldehyde hydrazone.

22. The aggregate particle of claim 17, wherein the zoospore attractant derivative is a condensation product of isovaleraldehdye and urea.

23. The aggregate particle of claim 17, wherein the binder is selected from the group consisting of proteins, albumins, natural latexes, man-made latexes, partially hydrolyzed polyvinyl alcohols, co-polymers of partially hydrolyzed polyvinyl alcohols, polyvinyl pyrrolidones, co-polymers of polyvinyl pyrrolidones, metal oxides, metal salts, gelatins, chitosans, starches, carbohydrates, amino acids and mixtures and derivatives thereof.

24. A method of controlling plant diseases caused by oomycete fungal pathogens including the steps of:
providing a formulation including the aggregate particle of claim 17;
diluting the formulation containing the aggregate particle of claim 17 in a suitable agricultural carrier; and
applying at least one of a formulation including the aggregate particle of claim 17 to at least one of a plant, an area adjacent to the plant, plant foliage, blossoms, stems, fruits, soil, seeds, germinating seeds, roots, liquid and solid growth media, and hydroponic growth solutions.

25. A method of controlling insects, plants diseases or weeds including the steps of:
providing a formulation including the aggregate particle of claim 17, and
applying an agriculturally effective amount of the formulation in mixture with at least one formulation of agricultural active ingredients and nutrients to at least one of a plant, plant foliage, blossoms, stems, fruits, an area adjacent to the plant, soil, seeds, germinating seeds, roots, liquid and solid growth media, and hydroponic growth solutions.
